(19) United States
(12) Patent Application Publication
(10) Pub. No.: US 2016/0096813 A1
(43) Pub. Date: Apr. 7, 2016

(54) TETRAHYDROFURAN-2,5-DICARBALDEHYDES (DIFORMYL-TETRAHYDROFURAN, DFTHF) AND PROCESS FOR MAKING THE SAME

(71) Applicant: ARCHER DANIELS MIDLAND COMPANY, Decatur, IL (US)

(72) Inventor: Kenneth Stensrud, Decatur, IL (US)

(21) Appl. No.: 14/892,072

(22) PCT Filed: Jun. 10, 2014
(86) PCT No.: PCT/US14/41665

§ 371 Date: Nov. 18, 2015

Related U.S. Application Data
(60) Provisional application No. 61/840,896, filed on Jun. 28, 2013.

(51) Int. Cl. C07D 307/24; C07D 307/14; C07D 307/12

(52) U.S. Cl. CPC C07D 307/24; C07D 307/12; C07D 307/14

(57) ABSTRACT

Tetrahydrofuran-(THF)-2,5-dicarbaldehyde and a process of preparing the same are described. The process involves reacting THF-diols in an inert organic solvent with an oxidizing agent at a temperature up to about 50 C. The process can use either HMF or THF-diols as starting materials, and enables a single-step conversion of THF-diols into a precursor material that can be transformed into a multitude of furanic derivative compounds. THF dicarbaldehyde can be modified according to certain reaction processes to generate either new or existing derivative compounds.

### PRIORITY CLAIM

The present application claims benefit of priority from U.S. Provisional Application No.: 61/840,896 filed Jun. 28, 2013, the contents of which are herein incorporated.

### FIELD OF INVENTION

The present invention relates to furanic carbaldehyde molecules, to particular methods by which such molecules are prepared, to certain derivative compounds or materials made from such molecules, and method for making certain derivative compounds.

### BACKGROUND

In recent years, an increasing effort has been devoted to find ways to utilize biomass as feedstock for the production of organic chemicals because of its abundance, renewability, and worldwide distribution. When considering possible downstream chemical processing technologies, the conversion of sugars to value-added chemicals is very important. Recently, the production of furan derivatives from sugars has become exciting in chemistry and in catalysis studies, because it aids major routes for achieving sustainable energy supply and chemicals production.

Cyclic bi-functional materials are useful as monomers in polymer synthesis and as intermediates generally. As these bi-functional materials are currently derived from increasingly scarce and costly petroleum resources, renewable source-based alternatives have been of increasing interest in recent years. Biomass contains carbohydrates or sugars (i.e., hexoses and pentoses) that can be converted into value added products. Production of biomass-derived products for non-food uses is a growing industry. Bio-based fuels are an example of an application with growing interest. Another application of interest is the use of biomass as feedstock for synthesis of various industrial chemicals from renewable hydrocarbon sources.

Carbohydrates represent the most abundant biologically-derived or renewable source feedstock for producing such alternative materials, but carbohydrates char easily and are generally unsuited to the high temperatures encountered in forming and processing the resultant polymer compositions. Further, compared to petroleum-based, hydrophobic aliphatic or aromatic feedstocks that have a low degree of functionalization, carbohydrates such as polysaccharides are complex, over-functionalized hydrophilic materials.

Consequently, researchers have sought to produce bio-based materials that derive from carbohydrates but which are less highly functionalized, for example, 2,5-furandicarboxylic acid (FDCA), levulinic acid and isosorbide, which could either serve as monomers and co-monomers or as intermediates in the synthesis of useful bio-based monomers and co-monomers.

As another important intermediate substance readily made from renewable resources, specifically carbohydrates, 2,5-(hydroxymethyl)furaldehyde (HMF, also 2,5-(hydroxymethyl)-furfural) is a renewable, monosaccharide-based building block.

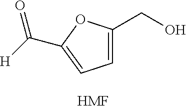

HMF is a suitable starting material for the formation of various furan ring derivatives that are known intermediates for a variety chemical syntheses, and as potential substitutes for benzene based compounds ordinarily derived from petroleum resources. Due to its various functionalities, it has been proposed that HMF could be utilized to produce a wide range of products such as polymers, solvents, surfactants, pharmaceuticals, and plant protection agents. As substitutes, one may compare derivatives of HMF to chemicals with the corresponding benzene-based rings or to other compounds containing a furan or tetrahydrofuran. HMF and 2,5-disubstituted furans and tetrahydrofuran derivatives, therefore, have great potential in the field of intermediate chemicals from renewable agricultural resources. In order to compete with petroleum based derivatives, however, preparation of HMF derivatives from common agricultural source materials, such as sugars, must be economical.

THF-diol, or 2,5-bis(hydroxymethyl)tetrahydrofuran, is another example of a bio-based material that has been of interest. Literature references, however, are relatively few in number. This may be due in part to the unavailability to date of HMF in commercial-scale quantities, from which THF-diol and derivatives of THF-diol would be prepared, although efforts have been long underway to develop a viable process for making HMF, see, e.g., U.S. Patent Application Publication No. 2009/0156841, Sanborn et al. Over the years, researchers have developed ways of transforming HMF into more easily used compounds such as 2,5-bis-(hydroxymethyl)-tetrahydrofuran (THF-diol) by means of reducing of HMF. Typically, THF-diol is prepared using Raney nickel reduction. An improvement on that approach is a method of preparing using a nickel and zirconium catalyst system, as described in U.S. Pat. No. 7,393,963 B2, Sanborn et al., the contents of which are incorporated herein by reference.

THF-diol is a rare yet versatile, organic compound, which has great potential as a starting material for various synthesis of plasticizers, resins, surfactants, pharmaceutical and agricultural chemicals. Due to its bi-functional reactivity from two -OH groups, THF-diol can be used as a precursor material in the area of polymers, such as polyurethanes (prepolymers, cast elastomers, thermoplastic elastomers, reaction injection molding and fibers such as spandex), polybutylene terephthalate (PBT), a large family of homopolymers and copolymers, and copolyester-ether thermoplastic elastomers.

Better and easier methods to utilize bio-based feedstock materials are warranted. The present invention can provide a pathway by which diformyltetrahydrofurans (DFTHF) can be derived facilely from HMF through THF-diol. As DFTHF are structurally analogous to another HMF derived molecular entity, 2,5-diformylfuran (DFF), which is well established as monomers to furan-based polymers and other materials (see e.g., Partenheimer, et al., Adv. Synth. Catal. 2001, 343, 102-111; Gandini, et al., Polym. Int. 1998, 4, 987; Baumtarden, et al., Chem. Eur. J. 1998, 4, 987, Xiang, et al., Polym. Int. 2013), this pathway can open a new way of addressing the need to make useful compounds from bio-based materials, which would be welcome in the growing, bio-based “green” chemicals industry.

### SUMMARY OF THE INVENTION

The present disclosure pertains, in part, to a process for preparing 2,5-diformyltetrahydrofurans (DFTHF) from either tetrahydrofuran (THF)-diols or 5-(hydroymethyl)-furfural (HMF). According to a first embodiment, the process involves providing a reaction mixture containing THF-diols and an inert organic solvent; reacting the THF-diols with an oxidizing agent at a reaction temperature up to about 50° C., to produce a THF-2,5-dialdehyde. The reaction can be performed in a non-inert atmosphere, such as air. The oxidizing agent exhibits selective reactivity with primary alcohol moieties. The oxidizing agent is not reactive with atmospheric oxygen or water vapor, and is inhibited from further oxidation of the resultant THF-2,5-dialdehyde. In another embodiment, the process includes first transforming HMF to THF-diols in a reduction step before selectively oxidizing according to the reaction above.

In another aspect, the present disclosure pertains to the diformyltetrahydrofurans (DFTHF) material produced via the foregoing process. THF-2,5-dicarbaldehyde is produced at a reaction yield of at least 60%, and after separation of the THF-2,5-dicarbaldehyde from by-products, an isolation yield of at least 50%. The THF-diols and THF-2,5-dicarbaldehyde in the resulting mixture are present in a 90:10 ratio of cis:trans diastereomers.

In another aspect, the present disclosure describes various derivative compounds that can be made from the THF-2,5-dicarbaldehyde as a starting or precursor material according to various chemical reactions available for organic synthesis. Such derivative materials can be useful as either substitutes for existing compounds or new chemical building blocks in various applications.

Additional features and advantages of the present synthesis process and material compounds will be disclosed in the following detailed description. It is understood that both the foregoing summary and the following detailed description and examples are merely representative of the invention, and are intended to provide an overview for understanding the invention as claimed.

### DETAILED DESCRIPTION OF THE INVENTION

### Section I—Description

When performing the complete oxidation of THF-diols to the carboxylic acid, the first stage oxidation product is THF dicarbaldehyde. THF dicarbaldehyde is a versatile compound that is open to various subsequent modifications. THF-dicarbaldehydes can open new pathways that enable a more efficient or easier and better use of HMF and/or THF-diols as starting materials and more convenient chemical synthesis. The present invention enables a single-step conversion process of THF-diols into a precursor material that can be transformed into a multitude of furanic derivative compounds.

A distinguishing and advantageous characteristic of the THF-dicarbaldehyde relative to isohexides or other bio-based asymmetric diols is the fixed chiral centers about the furan oxygen, which eliminates the potential for an inverted configuration. The fixed chiral centers at the alpha positions are not subject to inversions or reactivity as derivative chemistry will occur at the carbonyl moiety. Fixed stereochemistry is a desirable feature for functional control in synthetic application. Hence, THF-dicarbaldehydes are useful as precursor chemical materials for a variety of potential compounds, including for instance: pharmaceuticals or pharmaceutical precursor compounds, polymers or plastics, organic acids, solvents, rheology adjusters (e.g., surfactants, dispersants), etc.

### A. Preparation of THF-dicarbaldehyde

The present invention relates in part to a process of making tetrahydrofuran-(THF)-2,5-dicarbaldehyde. The process can use either HMF or THF-diols as starting materials. In an embodiment, the process involves: providing a reaction mixture containing THF-diols and an inert organic solvent, reacting the THF-diols with an oxidizing agent at a reaction temperature between about 10° C. to about 50° C., in a non-inert atmosphere.

In certain embodiments, the THF-diol can be derived desirably as a reduction product of HMF. When made from HMF, one is able to achieve a predominant diastereomeric ratio of the cis-species over the trans-species. One produces about a 90:10 ratio of cis:trans mixture of the THF-diol molecules. In contrast, when THF-diol is made from petrochemical sources the cis:trans molecules are in a racemic mixture (50:50). This feature of the present process allows for enhanced selectivity in the chirality of the reactive moieties of the THF-dicarbaldehyde molecule. Hence, an advantage of using HMF as the starting carbon source is a potential for a more facile separation after derivatization of the THF-dicarbaldehydes because of the predominance of the cis-species for a higher purity product.

Accordingly, a preliminary operation of converting HMF to THF-diols by reduction is performed before oxidizing the THF-diols, as outlined above. To generate THF-dialdehydes directly from HMF is impossible because of the double bonds in the HMF ring structure, as aldehyde groups in general will reduce much more readily than aromatic double bonds under any reduction condition. Hence, to generate THF-dialdehydes, one needs to first completely reduce HMF to THF-diols, and then selectively oxidize to form the dialdehyde, as depicted in Scheme 1.

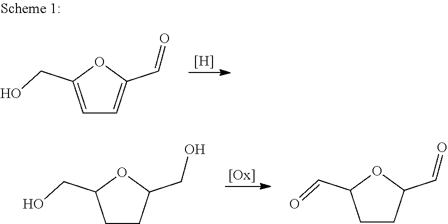

In converting THF-diol to the corresponding THF-2,5-dicarbaldehyde, the oxidizing agent performs a moderate, limited oxidation of the THF-diol hydroxyl groups in a single-step reaction. That is, the THF-diol and oxidizing agent should react spontaneously in a controlled, selective manner. The oxidizing agent exhibits selective reactivity with primary alcohol moieties, and is inhibited from further oxidizing the resultant THF-2,5-dialdehyde. Desirably, the oxidizing agent is non-toxic and is not reactive in air with atmospheric oxygen or water vapor. A minimum of one equivalent of oxidizing agent is consumed per hydroxyl (OH)-group of the THF-diols.

In certain embodiments, the reaction can be performed in a temperature range from about 12° C. or 15° C. to about 35° C. or 45° C. Typically, the reaction temperature is at ambient room temperature in a range from about 18° C. or 20° C. to about 25° C. or 28° C.

The oxidizing agent can be, for example, Dess Martin Periodinane (DMP). Other alternative synthesis processes may use pyridinium chloro-chromate (PCC) oxidation in which chromium is reduced from +6 to +3, or under Swern oxidizing conditions (dimethyl sulfoxide (DMSO), oxallyl-chloride). The Swern oxidation process however is not as favored, given that dimethyl-sulfide is one of the troubling by-products that one would need to treat and dispose of carefully when using a Swern oxidization protocol.

The present synthesis process can result in satisfactory yields of THF-dicarbaldehyde, as demonstrated in the accompanying Example 1. For instance, THF-2,5-dicarbaldehyde can be produced at a reaction yield of at least 60%, and can be separated from unreacted impurities or by-products for an isolation yield of at least 50%. In general, the process is able to produce THF-dicarbaldehyde in reasonably high molar yields of at least 50% from the THF-diol and/or HMF starting materials, typically about 55% to about 70% or 72%. With proper control of the reaction conditions and enhanced separation techniques (e.g., chromatography), one can achieve a yield of about 75%-80%-90% or better of the THF-dicarbaldehyde. HMF can be obtained either commercially or synthesized from relatively inexpensive, widely-available biologically-derived feedstocks.

### B. THF-Dicarbaldehyde Derivatives

In another aspect, the present disclosure pertains to certain furanic derivative compounds and methods for their preparation. The present THF dicarbaldehyde can be modified according to certain reaction processes to generate either new or conventionally produced derivative compounds from furan dicarbaldehyde. For example, it is envisioned that in subsequent reactions one can further oxidize the THF-dicarbaldehyde to its acid form and then use THF-dicarboxylic acid as a surrogate compound for p-terephthalate in polymerizations.

Once THF-dicarbaldehyde is synthesized according to the method as described, it can be transformed directly and readily to into other furanic derivative compounds by means of relatively straight-forward processes. For example, one can react the THF-dicarbaldehyde to perform at least one of the following reactions: 1) oxidation; 2) Schiff base (e g , imine) formation; 3) sulfonimidation, preceding reduction to sulfonamides (e.g., drug precursors); 4) synthesis of mono- and diacetals; 5) reductive amination; 6) Aldol condensation; 7) Aldol addition; 8) benzimidation, subsequent reductive debenzylation (e.g., bis-2,5-(amino-methyl)-THF): 9) Corey-Fuchs reaction (e.g., tetra-bromo-di-vinyl-THF); 10) formation of oxime, with subsequent reduction to substituted hydroxyl amines (e.g., bis(alkyl-hydroxylamines)); 11) Grignard addition, such as depicted conceptually in Scheme 2.

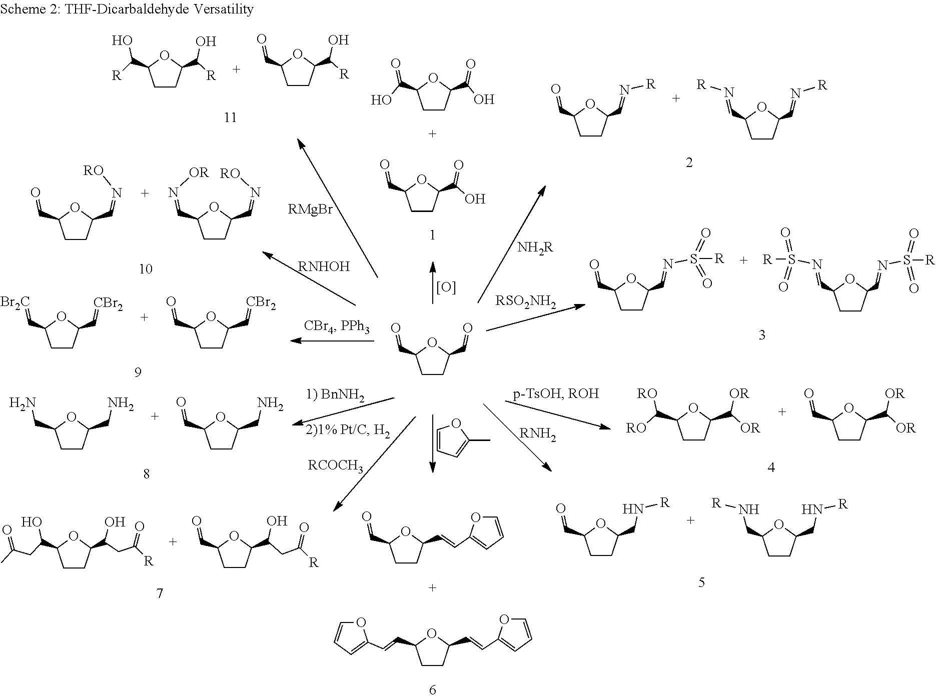

wherein [O] is oxidation, R═H, alkyl, alkenyl, alkynyl, or aryl species. Other reactions, such as Wittig reactions (glide addition, elimination) as demonstrated in the accompanying examples, can also be used to generate derivative compounds from the THF-dicarbaldehyde.

Table 1 presents some illustrative examples of particular furanic derivative compounds that can be made from each type of reaction depicted in Scheme 2. These examples are intended to be non-limiting, and analogue compounds are also contemplated.

TABLE 1

| Reaction | General Structure | Example IUPAC Name | Example Structure |
|---|---|---|---|
| Oxidation | | (2R,5S)-tetrahydrofuran- 2,5-dicarboxylic acid | 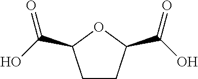 [SMILES: O=C(O)[C@@H]1CC[C@H](C(=O)O)O1] |
| Schiff Base formation | 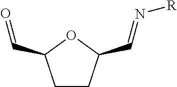 | (2S,5R)-5-((E)- (phenylimino)methyl)- tetrahydrofuran-2- carbaldehyde | 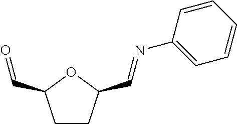 [SMILES: O=C[C@@H]1CC[C@H](/C=N/c2ccccc2)O1] |
| | 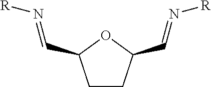 | (N,N′E,N,N,E)- N,N′-(((2R,5S)- tetrahydrofuran-2,5-diyl)- bis(methanylylidene))- dianiline | 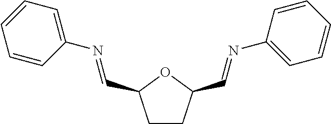 [SMILES: C(=N\c1ccccc1)/[C@@H]1CC[C@H](/C=N/c2ccccc2)O1] |
| Sulfon- imidation | 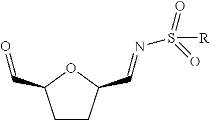 | (E)-N-(((2R,5S)-5- formyltetrahydrofuran- 2-yl)methylene)- ethanesulfonamide | 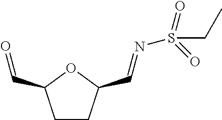 |
| | 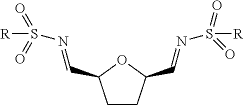 | (N,N′E,N,N′E)- N,N′-(((2R,5S)- tetrahydrofuran-2,5-diyl)- bis(methanylylidene))- diethanesulfonamide | 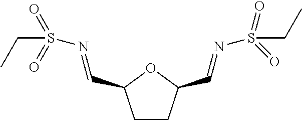 [SMILES: CCS(=O)(=O)/N=C/[C@@H]1CC[C@H](/C=N/S(=O)(=O)CC)O1] |
| mono- & diacetals synthesis | 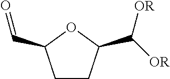 | (2S,5R)-5- (dimethoxymethyl)- tetrahydrofuran- 2-carbaldehyde | 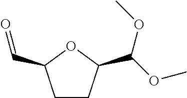 |
| | 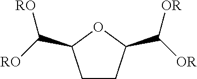 | (2R,5S)-2,5- bis(dimethoxymethyl)- tetrahydrofuran | 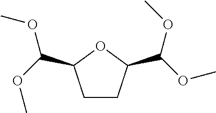 |
| reductive animation | 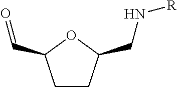 | (2S,5R)-5- ((isobutylamino)methyl)- tetrahydrofuran- 2-carbaldehyde | 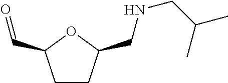 |
| | 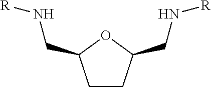 | N,N′-(((2R,5S)- tetrahydrofuran-2,5- diyl)bis(methylene))bis(2- methylpropan-1-amine) | 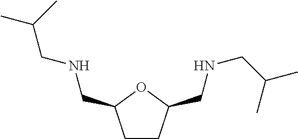 [SMILES: CC(C)CNC[C@H]1CC[C@@H](CNCC(C)C)O1] |
| | | N,N′-(((2R,5S)- tetrahydrofuran-2,5- diyl)bis(methylene))bis (propan-1-amine) | 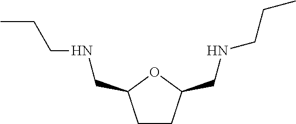 |
| Aldol condensation | | (2S,5R)-5-((E)-2- (furan-2-yl)- vinyl)tetrahydrofuran- 2-carbaldehyde | 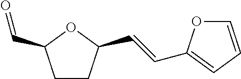 [SMILES: O=C[C@@H]1CC[C@H](/C=C/c2ccco2)O1] |
| | | (2R,5S)-2,5-bis((E)- 2-(furan-2-yl)- vinyl)tetrahydrofuran | 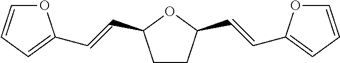 [SMILES: C(=C\[C@@H]1CC[C@H](/C=C/c2ccco2)O1)/c1ccco1] |
| | | (2E,2′E)-4,4′-((2R,5S)- tetrahydrofuran- 2,5-diyl)bis(1-phenylbut- 2-en-1-one) | 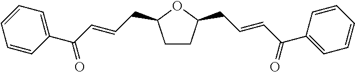 [SMILES: O=C(/C=C/C[C@@H]1CC[C@H](C/C=C/C(=O)c2ccccc2)O1)c1ccccc1] |
| Aldol addition | 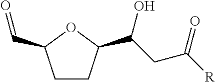 | (2S,5R)-5-((R)-1- hydroxy-3-oxo-3- phenylpropyl) tetrahydrofuran-2- carbaldehyde | 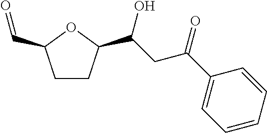 [SMILES: O=C[C@@H]1CC[C@H](C(O)CC(=O)c2ccccc2)O1] |
| | 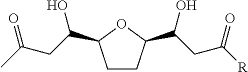 | (3S,3′R)-3,3′-((2R,5S)- tetrahydrofuran- 2,5-diyl)bis(3-hydroxy- 1-phenylpropan-1-one) | 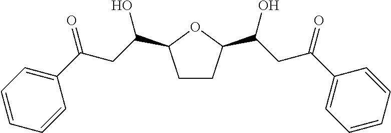 [SMILES: O=C(CC(O)[C@@H]1CC[C@H](C(O)CC(=O)c2ccccc2)O1)c1ccccc1] |
| benzimidation | | (2S,5R)-5-(aminomethyl)- tetrahydrofuran- 2-carbaldehyde | 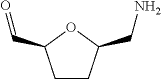 |
| | | ((2R,5S)-tetrahydrofuran- 2,5-diyl)- dimethanamine | 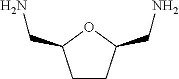 |
| Corey-Fuchs reaction | | (2S,5R)-5- (2,2-dibromovinyl)- tetrahydrofuran- 2-carbaldehyde | 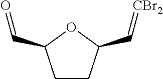 |
| | | (2R,5S)-2,5-bis(2,2- dibromovinyl)- tetrahydrofuran | 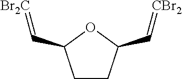 |
| oximation | 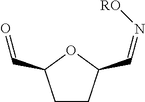 | (2S,5R)-5-((Z)- (ethoxyimino)methyl)- tetrahydrofuran- 2-carbaldehyde | 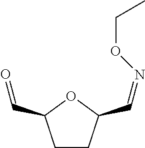 |
| | 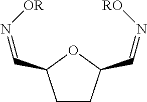 | (1Z,1′Z)-5-((Z)- (ethoxyimino)methyl)- tetrahydrofuran-2- carbaldehyde O-ethyl oxime | 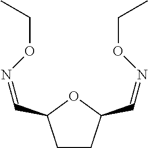 |
| | | (1E,1′E)-5-((E)- (hydroxyimino)- methyl)tetrahydrofuran- 2-carbaldehyde oxime | 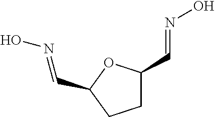 [SMILES: O/N=C/[C@@H]1CC[C@H](/C=N/O)O1] |
| Grignard addition | 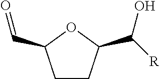 | (2S,5R)-5-((R)- 1-hydroxypropyl)- tetrahydrofuran- 2-carbaldehyde | 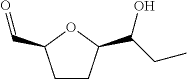 |
| | 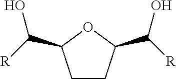 | (1S,1′R)-1,1′-((2R,5 S)- tetra-hydrofuran- 2,5-diyl)bis(propan-1-ol) | 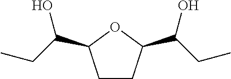 |

The foregoing list of reactions and the examples are not intended to be an exhaustive catalogue of derivative compounds, but merely a non-limiting illustration of representative derivatives. Section II, below, presents other examples of furanic derivative compounds that can be synthesized from the present THF-dicarbaldehyde.

### Section II.—Examples

### A—THF-Dicarbaldehyde Preparation

### Example 1

The following is an example of a scheme for synthesizing THF-2,5-dialdehydes C (cis) and D (trans).

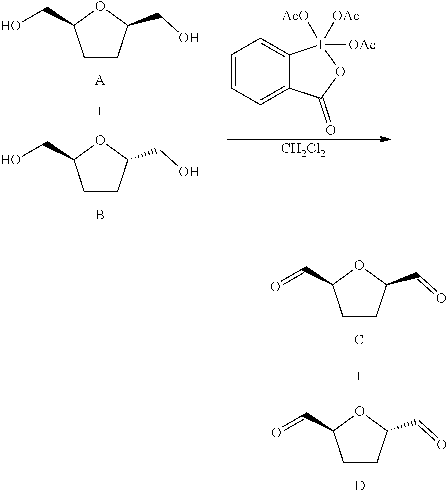

Experimental: A 50 mL round bottomed flask, equipped with a tapered PTFE coated magnetic stir bar, was charged with 100 mg of THF diols (9:1 dr A to B, 0.756 mmol), 704 mg of Dess Martin Periodinane (DMP, 1.67 mmol), and 25 mL of anhydrous methylene chloride. The mixture was stirred at room temperature (˜20-23°) for 24 hours. After this time, a profusion of white solid was observed, which was removed by filtration. The filtrate was then poured directly on a pre-fabricated silica gel column where flash chromatography using a 5:1 to 1:1 hexanes:ethyl acetate gradient manifested C and D (and diastereomers) with an R_{f} of 0.48, and weighing 51 mg after solvent evaporation in vacuo (52% of theoretical). ¹H NMR (400 MHz, CDCl₃) δ (ppm) C: 9.70 (s, 2H), 4.62 (m, 2H), 2.20 (m, 2H), 2.00 (m, 2H). D: 9.76 (s, 1H), 9.72 (s, 1H), 4.57 (m, 2H), 2.17 (m, 2H), 1.97 (m, 2H). ¹³C NMR (125 MHz, CDCl₃) C: 201.24, 94.04, 23.33. D: 200.99, 92.72, 22.81.

### B—Derivatives of THF-Dicarbaldehyde

In the following examples, the predominant (cis) isomer (˜90%) is presented in the reaction schemes; nonetheless, it is understood that trans isomers will be also present in the final product at about 10%.

### Example 2

### Aldol condensation: Synthesis of (3E ,3′E)-4,4′-((2R,5S)-tetrahydrofuran-2,5-diyl)bis(but-3-en-2-one), B.

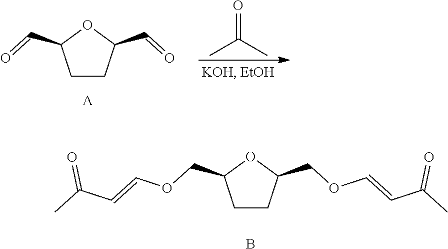

Experimental: A 10 mL round bottomed flask equipped with a Y4^{″} PTFE coated magnetic stir bar was charged with 100 mg of A (0.780 mmol), 175 mg of KOH (3.12 mmol), 573 μL of acetone (7.80 mmol, 10 eq.), and 5 mL anhydrous ethanol. The mixture was stirred for 24 h at room temperature. After this time, an aliquot was removed (˜1 mL): One spotted on a normal phase TLC plate, which indicated a single spot Rf=0.61, cerium molybdate stain) after development with a 10% hexanes in ethyl acetate mobile phase. The absence of signature band of A, Rf=0.49, was patently absent, specifying full conversion of this reagent. The second aliquot was diluted with deuterated acetone and examined by ¹H NMR (400 MHz), manifesting no characteristic aldehyde resonance frequencies and thus corroborating that A had fully converted. The mixture was then transferred to a 125 mL beaker and diluted with 25 mL volumes of methylene chloride and water, then transferred to a 125 mL separatory funnel The organic phase was removed, aqueous phase extracted x3 with 5 mL methylene chloride, and combined organic phases dried with anhydrous sodium sulfate and concentrated in vacuo, producing 142 mg of B as a pale yellow semi-solid (88% of theoretical). ¹H NMR (400 MHz, CDCl₃) δ (ppm) 6.99 (m, 2H) 6.12 (d, J=13.6 Hz, 2H), 4.61 (m, 2H), 2.42 (s, 6H), 2.02 (m, 2H), 1.92 (m, 2H); ¹³C NMR (100 MHz, CDCl₃), δ (ppm) 195.6, 148.2, 129.6, 86.1, 26.1, 19.9

### Example 3

Wittig reaction (glide addition, elimination): Synthesis of (2E,2′E)-dimethyl 3,3′-((2R,5S)-tetrahydrofuran-2,5-diyl)diacrylate, B.

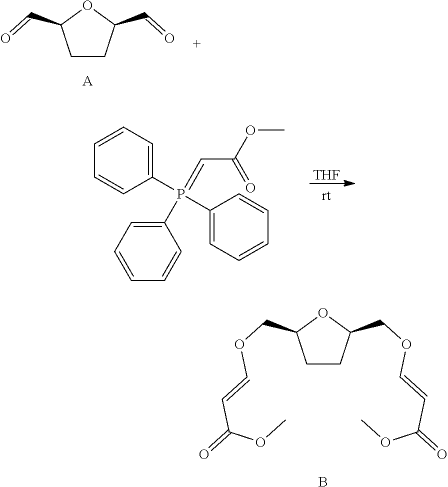

Experimental: A 10 mL round bottomed flask equipped with a Y4^{″} PTFE magnetic stir bar was charged with 100 mg of A (0.780 mmol), 523 mg of methyl-(triphenylphosphoranylidene)acetate (1.56 mmol), and 5 mL of anhydrous THF. The mixture was stirred at room temperature overnight. After this time, an aliquot was removed (˜1 mL): One spotted on a normal phase TLC plate, which indicated two spots Rf₁=solvent front, UV-Vis illumination (triphenyphosphine oxide), Rf₂=0.55, cerium molybdate stain) after development with a 10% hexanes in ethyl acetate mobile phase. The absence of signature band of A, Rf=0.49, was patently absent, specifying full conversion of this reagent. The second aliquot was diluted with deuterated acetone and examined by ¹H NMR (400 MHz), manifesting no characteristic aldehyde resonance frequencies and thus corroborating that A had fully converted. The mixture was then transferred to a 125 mL beaker and diluted with 25 mL volumes of methylene chloride and water, then transferred to a 125 mL separatory funnel The organic phase was removed, aqueous phase extracted x3 with 5 mL methylene chloride, and combined organic phases dried with anhydrous sodium sulfate and concentrated in vacuo, producing 140 mg of B as a loose colorless oil (76% of theoretical). ¹H NMR (400 MHz, CDCl₃) δ (ppm) 6.86 (m, 2H) 6.01 (d, J=13.0 Hz, 2H), 4.50 (m, 2H), 3.81 (s, 6H), 2.01 (m, 2H), 1.90 (m, 2H); ¹³C NMR (100 MHz, CDCl₃), δ (ppm) 165.1, 147.4, 125.0, 83.9, 50.7, 19.4.

### Example 4

Grignard reaction: Synthesis of ((1S,1′R)-1,1′-((2R,5S)-tetrahydrofuran-2,5-diyl)bis(but-3-en-1-ol) B, and diastereomer C.

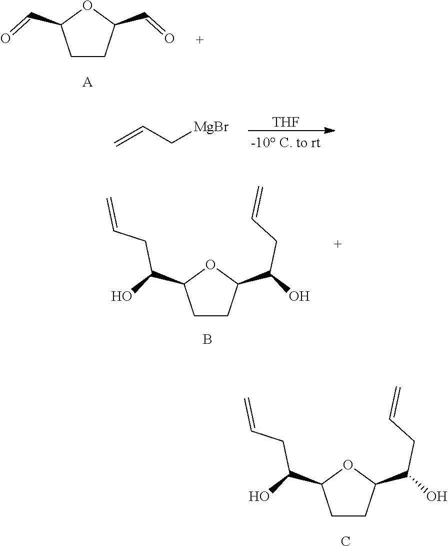

Experimental: A single-neck, oven dried, 10 mL round bottomed flask equipped with a ¼″ PTFE magnetic stir bar was charged with 100 mg of A (0.780 mmol) and 5 mL of anhydrous THF. The neck was then stoppered with a rubber septum and an argon gas inlet attached. The flask was then immersed in an ice/brine bath (˜10° C.), and, while vigorously stirring and under an argon blanket, 1.56 mL of allylmagnesium bromide (1 M in diethyl ether, 1.56 mmol) was added dropwise over 10 minutes. The brine was then removed and mixture continued stirring overnight at room temperature overnight. After this time, the solution was diluted with 10 mL of methylene chloride and 10 mL of water and resultant biphasic mixture transferred to a separatory funnel The bottom layer was partitioned, and aqueous layers extracted twice with 5 mL volumes of methylene chloride. The organic layers were then combined, dried with anhydrous sodium sulfate and concentrated under reduced pressure, producing 121 mg of B and C as a light-yellow, loose oil (73% of theoretical). ¹H NMR (400 MHz, CDCl₃) δ (ppm) 5.81 (m, 2H) 5.02 (m 4H), 4.55 (m, 2H), 3.62 (m, 2H), 3.55 (m, 2H), 2.22 (m, 2H), 2.01 (m, 2H), 1.88 (m, 2H), 1.79 (m, 2H); ¹³C NMR (100 MHz, CDCl₃), δ (ppm) 133.3, 117.0, 84.1, 77.3, 39.4, 29.7.

The generation of immediately adjacent chiral centers potentially opens the way to multiple stereoisomers (e.g., 2⁴=16 possible structures).

### Example 5

Reductive amination: Synthesis of 2,2′-((((((2R,5S)-tetrahydrofuran-2,5-diyl)-bis(methylene))bis(azanediyl))bis(ethane-2,1-diyl))bis(azanediyl))diethanol, B.

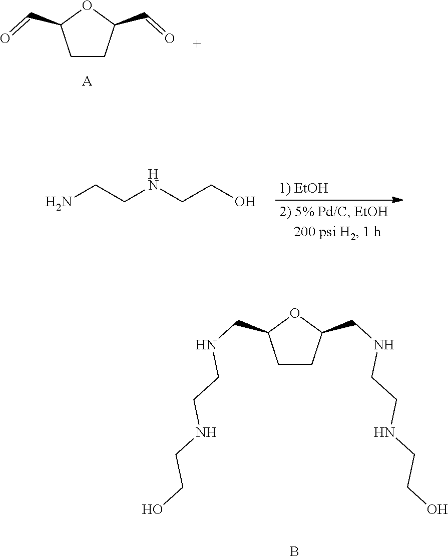

Experimental: A single-neck, 10 mL round bottomed flask equipped with a ¾″ PTFE magnetic stir bar was charged with 100 mg of A (0.780 mmol), 158 μL of aminoethylethanolamine (AEEA, 1.56 mmol), and 5 mL of absolute ethanol. A condenser was then attached to the neck and mixture brought to reflux for 4 h. After this time, an aliquot was removed, analyzed by ¹H NMR (400 MHz, CDCl₃), which disclosed the absence of aldehyde signals specific (˜9.7 ppm) to A. The mixture was then cooled to room temperature, transferred to a 75 mL Parr vessel, along with 500 mg of 5% Pd/C. After being made hermetic, the vessel was charged with H₂ until the pressure gauge read 200 psi, and overhead stirring begun. An aliquot was removed after 1 h and analyzed by ¹H NMR (400 MHz, CDCl₃), which revealed no signals characteristic of the imine intermediate (˜7.7 ppm). The mixture was then filtered and concentrated under reduced pressure, furnishing 227 mg of a colorless oil (96% of theoretical). ¹H NMR (400 MHz, CDCl₃) δ (ppm) 3.92 (m, 2H), 3.65 (m, 2H), 3.44 (t, J=6.2 Hz, 4H), 2.85 (m, 2H), 2.77 (m, 4H), 2.55-2.50 (m, 10H), 2.01 (m, 2H), 1.71 (m, 2H); ¹³C NMR (100 MHz, CDCl₃), δ (ppm) 82.5, 63.4, 52.0, 51.5, 50.6, 49.7, 33.5.

### Example 6

Heyns oxidation: Synthesis of (2R,5S)-tetrahydrofuran-2,5-dicarboxylic acid, B.

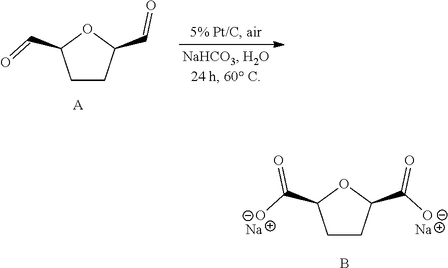

Experimental: A single-neck, 100 mL round bottomed flask equipped with a magnetic stir bar was charged with 1.00 g of A (7.80 mmol), 1.61 g of 5% Pt/C (200 g/mol HMF), 3.99 g of NaHCO₃ (47.6 mmol) and 60 mL of deionized water. The neck of the flask was then capped with a rubber septum and an air inlet affixed via an 18 gauge stainless needle whose beveled tip was positioned near the bottom of the heterogeneous solution. In addition, six 2 inch, 16 gauge needles pierced the septum, utilized as air vents. While stirring, the flask was immersed in an oil bath and heated at 60° C. with vigorous sparging of air for a 24 hour time period. After this time, the Pt/C was removed by filtration and the aqueous residue analyzed by silica gel thin layer chromatography using a 20% methanol in ethyl acetate developing solution and UV light for spot illumination. A single band, positioned at the baseline, was observed while that for HMF (0.90 with an authentic sample) was absent, suggesting that A had been fully converted to the disodium salt, B. Additionally, ¹H NMR analysis (400 MHz, D₂O) of the product mixture failed to descry the characteristic aldehyde signal of A (˜9.6 ppm). Cogent proof for the presence of the disodium salt of B arose from ¹³C NMR (D₂O, 125 MHz), where only three signals at 171.33, 86.8, 31.3 ppm were observed.

Although the present invention has been described generally and by way of examples, it is understood by those persons skilled in the art that the invention is not necessarily limited to the embodiments specifically disclosed, and that modifications and variations can be made without departing from the spirit and scope of the invention. Thus, unless changes otherwise depart from the scope of the invention as defined by the following claims, they should be construed as included herein.

1. A process of making a tetrahydrofuran-(THF)-2,5-dicarbaldehyde, comprising:
providing a reaction mixture containing THF-diols and an inert organic solvent; reacting said THF-diols with an oxidizing agent at a reaction temperature between about 10° C. to about 50° C., in a non-inert atmosphere.

2. (canceled)

3. The process according to claim 1, wherein said oxidizing agent exhibits selective reactivity with primary alcohol moieties.

4. The process according to claim 1, wherein said oxidizing agent is not reactive with atmospheric oxygen or water vapor.

5. The process according to claim 1, wherein said oxidizing agent is inhibited from further oxidation of said THF-2,5-dialdehyde.

6. The process according to claim 1, wherein said oxidizing agent is non-toxic.

7. The process according to claim 1, wherein said reaction temperature is from about 12° C. to about 45° C.

8. The process according to claim 1, wherein a minimum of one equivalent of oxidizing agent is consumed per hydroxyl (OH)-group of said THF-diols.

9. The process according to claim 1, wherein said oxidizing agent is at least one of the following: Dess Martin Periodinane (DMP), Swern oxidation agent (DMSO, oxallyl-chloride), pyridinium chloro-chromate (PCC).

10. The process according to claim 1, wherein said THF-diols and THF-2,5-dicarbaldehyde are present in a 90:10 ratio of cis:trans diastereomers.

11. The process according to claim 1, wherein said THF-2,5-dicarbaldehyde is produced at a reaction yield of at least 60%.

12. The process according to claim 1, further comprising separating said THF-2,5-dicarbaldehyde from by-products to have an isolation yield of at least 50%.

13. A tetrahydrofuran (THF)-2,5-dicarbaldehyde:

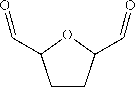

14. The THF-2,5-dicarbaldehyde according to claim 13, wherein said THF-2,5-dicarbaldehyde is about 90% ((2R,5S)-tetrahydrofuran-2,5-dicarbaldehyde) 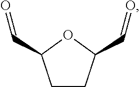
and about 10% (2R,5R)-tetrahydrofuran-2,5-dicarbaldehyde 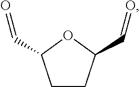
and (2S,5S)-tetrahydrofuran-2,5-dicarbaldehyde

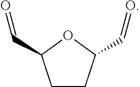

15. A method of preparing a furanic derivative compound of a tetrahydrofuran (THF)-2,5-dialdehyde comprising: reacting a mixture containing THF-diols and an inert organic solvent with an oxidizing agent at a reaction temperature up to about 50° C., in a non-inert atmosphere; and transforming said THF-dicarbaldehyde into a furanic derivative compound.

16. (canceled)

17. The method according to claim 15, wherein said transforming to said furanic derivative compound comprises: performing least one of the following reactions: 1) oxidation; 2) Schiff base modification; 3) sulfonimidation, preceding reduction to sulfonamides; 4) synthesis of mono- and diacetals; 5) reductive amination; 6) Aldol condensation; 7) Aldol addition; 8) benzimidation, subsequent reductive debenzylation: 9) Corey-Fuchs reaction; 10) formation of oxime, with subsequent reduction to substituted hydroxyl amines; 11) Grignard addition; and 12) Wittig reaction.

18. The method according to claim 15, wherein said furanic derivative compound consists predominantly of a single isomer species.

19. The method according to claim 15, wherein said furanic derivative compound consists of about 90% of cis isomers and about 10% trans isomers.

20. (canceled)

21. (canceled)

22. (canceled)

23. (canceled)

24. (canceled)

25. (canceled)

26. (canceled)

27. (canceled)

28. (canceled)

29. A furanic derivative compound transformed according to the anyone of the following reactions: 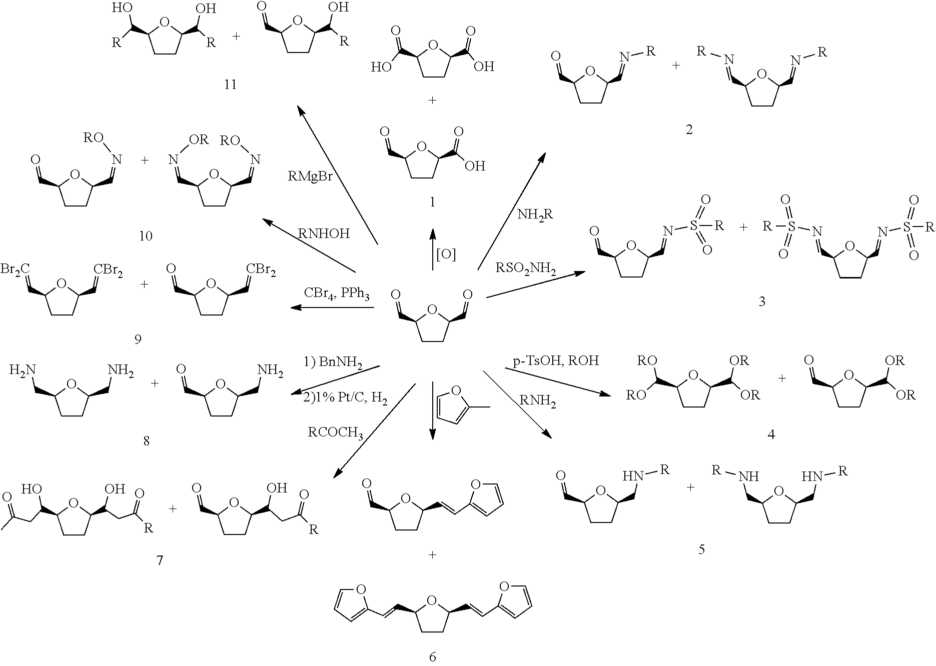
wherein [O] is oxidation, R′H, alkyl, alkenyl, alkynyl, or aryl species.

30. The furanic derivative compounds according to claim 29, wherein said furanic derivative compound is at least one of the following:

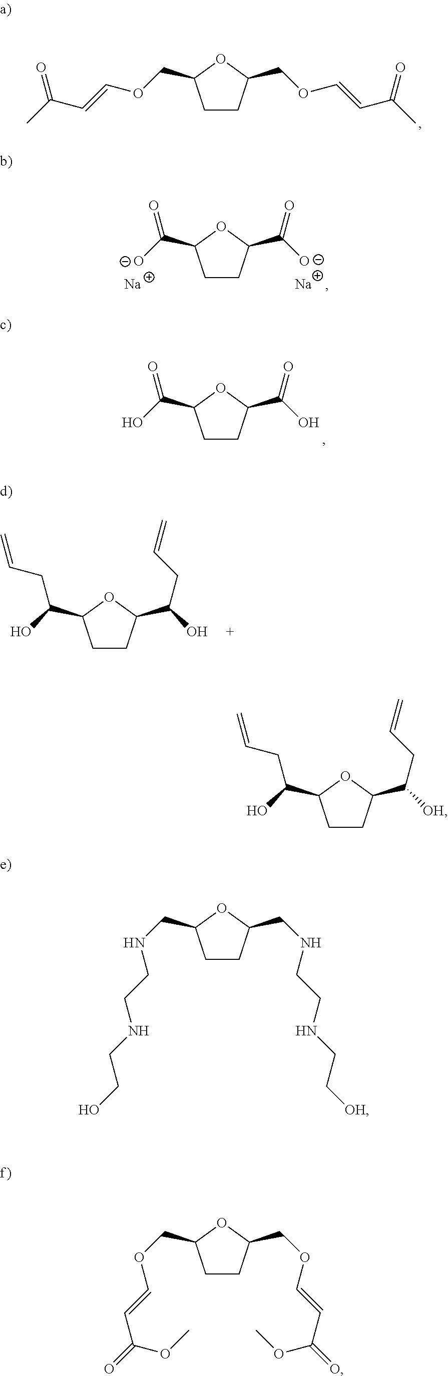

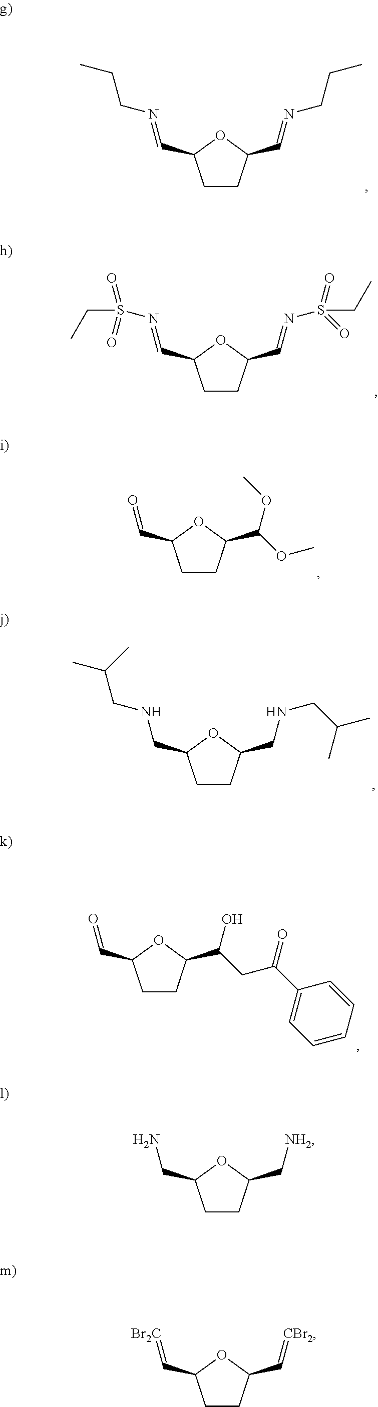

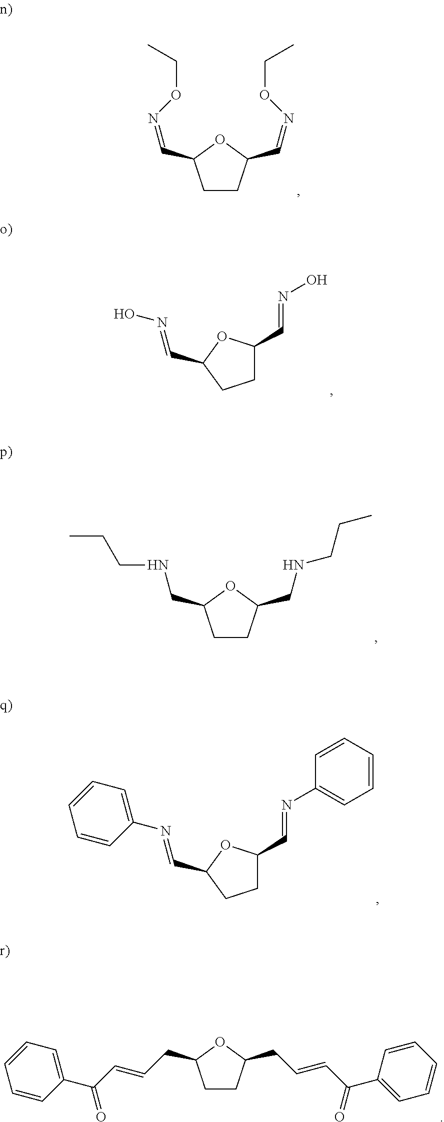 [SMILES: CCO/N=C\[C@@H]1CC[C@H](/C=N\OCC)O1 ; O/N=C/[C@@H]1CC[C@H](/C=N/O)O1 ; CCCNC[C@H]1CC[C@@H](CNCCC)O1 ; C(=N\c1ccccc1)/[C@@H]1CC[C@H](/C=N/c2ccccc2)O1 ; O=C(/C=C/C[C@@H]1CC[C@H](C/C=C/C(=O)c2ccccc2)O1)c1ccccc1]

31. A furanic derivative compound transformed by at least one of the following: a) oxidation; b) Schiff-base modification, having a general formula: 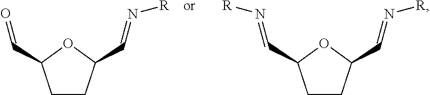
where R is a H, alkyl, alkenyl, alkynyl, or aryl species; c) sulfonimidation, having a general formula: 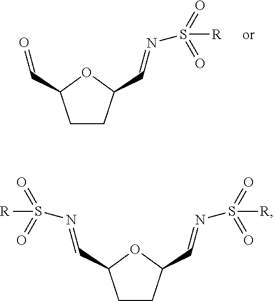
where R is a H, alkyl, alkenyl, alkynyl, or aryl species; d) synthesis of mono- and diacetals, having a general formula: 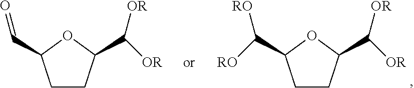
where R is a H, alkyl, alkenyl, alkynyl, or aryl species; e) reductive amination, having a general formula 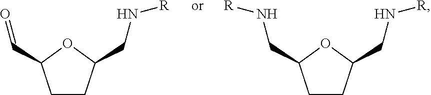
where R is a H, alkyl, alkenyl, alkynyl, or aryl species; f) Aldol condensation, having a general formula: 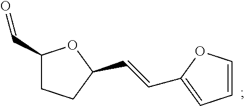
g) Aldol addition, having a general formula: 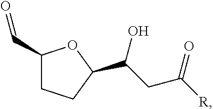
where R is a H, alkyl, alkenyl, alkynyl, or aryl species; h) formation of oxime, having a general formula: 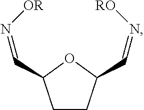
where R is a H, alkyl, alkenyl, alkynyl, or aryl species; or i) a Wittig reaction.
